(19) United States
(12) Patent Application Publication
(10) Pub. No.: US 2022/0117780 A1
(43) Pub. Date: Apr. 21, 2022

(54) SMART AUXILIARY DIAGNOSIS SYSTEM AND METHOD FOR FUNDUS OCULI LASER SURGERY

(71) Applicant: Brightview Medical Technologies (Nanjing) Co. Ltd., Nanjing (CN)

(72) Inventor: Jie ZHANG, Nanjing (CN)

(21) Appl. No.: 17/428,188

(22) PCT Filed: May 29, 2019
(86) PCT No.: PCT/CN2019/088979

§ 371 Date: Aug. 3, 2021

(30) Foreign Application Priority Data
May 24, 2019 (CN) 201910442076.2

(51) Int. Cl. A61F 9/009; A61B 3/10; A61B 3/12; A61B 3/14; G16H 15/00; G16H 50/20

(52) U.S. Cl. CPC A61F 9/009; A61B 3/1025; A61B 3/12; A61F 2009/00851; A61B 3/14; G16H 15/00; G16H 50/20; A61B 3/102

(57) ABSTRACT

Disclosed are a smart auxiliary diagnosis system and method for fundus oculi laser surgery, comprising a imaging stabilization and laser treatment device (1), a data control device (2), an image display device (3), and a data processing device (4); a first database (41) thereof stores fundus oculi image data; disease feature data in a fundus oculi image is extracted by means of a feature extraction module (42); a data analysis matching module (45) is used to perform a comparison operation, perform matching with disease feature data stored in a known-case feature template library (44), and store the result of the matching operation in a second database (43); if the degree of matching exceeds a set threshold, then a corresponding auxiliary diagnosis conclusion is provided, and an auxiliary diagnosis report is generated by means of a diagnosis report generation module (46).

### FIELD OF THE INVENTION

The present invention relates to diagnosis and treatment technology for fundus laser surgery, in particular to a smart auxiliary diagnosis system and method for fundus laser surgery.

### BACKGROUND

Diabetic retinopathy (DR) is the first blinding disease among working-age people. The main causes of visual impairment and blindness in DR patient are proliferative diabetic retinopathy (PDR) and diabetic macular edema (DME), and laser photocoagulation is the main treatment method for the patient with diabetic retinopathy (DR).

The current fundus laser treatment technology for the patient with diabetic retinopathy (DR), macular degeneration and other ophthalmic diseases mainly depends on a clinician manually operating a laser for fixed-point strikes, or using a two-dimensional galvanometer (vibrating mirror) for treatment by an array-shaped laser strike. However, these technologies are often not accurate enough in use, and treatment measures are based on a mechanical contact. There are usually defects that an operation time is long and an experience of the clinician and patient is poor (such as aggravated DME causing side effects such as a permanent central vision damage, laser scar enlargement, etc., which cause a peripheral vision decline, visual field reduction, and scotopic vision deficiency of the patient). In addition, the existing methods of manual fundus laser surgery or treatment by a dot-array laser strike with a scanning galvanometer mainly depend on judgment and operation experience of the clinician, and may not be automated and smart for preoperative diagnosis and implementation of laser fundus surgery. Therefore, the efficiency of diagnosis and treatment is not high, and there is a certain surgical treatment risk. It is not suitable for the clinician with insufficient clinical diagnosis and treatment experience and has an obvious limitation.

### SUMMARY OF THE INVENTION

In view of this, the main objective of the present invention is to provide a smart auxiliary diagnosis system and method for fundus laser surgery, so as to solve the problem that a process of the existing preoperative diagnosis and treatment for fundus laser surgery highly depends on the judgment and operation experience of the clinician, which makes the implementation of surgical treatment difficult. By using the auxiliary diagnosis system, an auxiliary diagnosis report such as a preoperative diagnosis scheme, intraoperative target determination and postoperative effect prediction may be automatically provided, a misdiagnosis rate may be reduced and the process of diagnosis and operation by the clinician may be further simplified. While an accuracy of the surgical treatment is ensured, an diagnostic efficiency may be improved, and a risk of laser surgery is greatly reduced.

To achieve the above objective, a technical solution of the present invention is as follows.

A smart auxiliary diagnosis system for fundus laser surgery includes a imaging stabilization and laser treatment device 1, a data control device 2, and an image display device 3; and further includes a data processing device 4.

The data processing device includes a first database 41, a feature extraction module 42, a data analysis matching module 43, a case feature template library 44, a second database 43, and a diagnosis report generation module 46; the first database 41 is used to store high-definition fundus image data collected by the imaging stabilization and laser treatment device 1 at any angle and with various imaging methods; disease feature data in the fundus image is extracted by the feature extraction module 42, and compared with known disease feature data stored in the case feature template library 44 by a comparison operation using the data analysis matching module 45, and a matching operation result is stored in the second database 43, if a matching degree exceeds a set threshold, a corresponding auxiliary diagnosis conclusion is provided, and then an auxiliary diagnosis report is generated through the diagnosis report generation module 46.

The imaging stabilization and laser treatment device 1 comprises:

the imaging diagnosis module for obtaining a reflection signal returned from the fundus at any angle in real time or/and obtaining image data of the fundus;

the laser treatment module for tracking and locking a fundus target in real time, and automatically adjusting a laser dose output.

The imaging diagnosis module supports one or more of a confocal scanning laser ophthalmoscope SLO, a line scanning ophthalmoscope LSO, a fundus camera, or an adaptive optics scanning light ophthalmoscope AOSLO.

The imaging diagnosis module further supports a combination of a plurality of imaging forms, including one or more of SLO+OCT, fundus camera+OCT, fundus camera+SLO or AOSLO+SLO.

The smart auxiliary diagnosis system for fundus laser surgery further includes a deep learning module 47 for performing a large amount of data training by combining the collected fundus image of a patient with the disease feature data extracted from the fundus image, and obtaining the matching operation result for a medical expert's reference by automatically performing a data analysis matching operation.

It further includes: processing the matching operation result for the medical expert's reference:

matching the matching operation result of which the matching degree is greater than the set threshold with a case in the case feature template library and register it as a case; or,

writing case feature data corresponding to the fundus image with the matching operation result of which the matching degree is less than the set threshold confirmed by the medical expert into a new case feature template for inputting into the case feature template library 44, that is, updating the case feature template library.

Contents of the auxiliary diagnosis report include a preoperative diagnosis scheme, an intraoperative target determination scheme, and a postoperative treatment effect prediction result.

A smart auxiliary diagnosis method for fundus laser surgery includes the following steps:

A. collecting high-definition fundus image data at any angle and with various imaging methods a using imaging stabilization and laser treatment device 1, and storing it in a first database 41 of a data processing device 4;

B. extracting disease feature data in the fundus image by a feature extraction module 42, and perform a comparison operation using a data analysis matching module 45 to obtain a comparison result;

C. matching the comparison result with the disease feature data stored in known case feature template library 44, and storing a matching operation result in a second database 43;

D. if a matching degree exceeds a set threshold, providing a corresponding auxiliary diagnosis conclusion and then generating an auxiliary diagnosis report by a diagnosis report generation module 46.

After step D, it further includes:

E. by a deep learning module 47, performing a large amount of data training by combining the collected fundus image of a patient with the disease feature data extracted from the fundus image, and obtaining the matching operation result for a medical expert's reference by automatically performing a data analysis matching operation.

Step E further includes:

E1. matching the matching operation result of which the matching degree is greater than the set threshold with a case in the case feature template library and register it as a case; or,

E2. writing case feature data corresponding to the fundus image with the confirmed matching operation result of which the matching degree is less than the set threshold into a new case feature template for inputting into the case feature template library 44, that is, updating the case feature template library.

The smart auxiliary diagnosis system and method for fundus laser surgery of the present invention have the following beneficial effects:

1) The smart auxiliary diagnosis system and method for fundus laser surgery of the present invention may not only provide a visualized smart diagnosis and treatment reference scheme for fundus laser surgery of the patient, but also provide real-time human fundus image collection, real-time disease analysis and treatment reference area planning, and adaptive adjustment of laser dose, automatic laser treatment; and may also support laser treatment in the mode of manual intervention.

2) The smart auxiliary diagnosis system for fundus laser surgery of the present invention integrates a variety of ophthalmic fundus imaging technologies and laser treatment technologies, and may realize a one-stop diagnosis plus treatment service, and meanwhile, may realize an intelligent, automated, and high accurate treatment, and simplify the operation to improve the patient's experience.

3) The treatment device for fundus laser surgery of the present invention may integrate a laser treatment function through a mechanical device and share hardware with an imaging device, which has a characteristics of cost saving.

4) The treatment device for fundus laser surgery of the present invention also provides a variety of imaging diagnostic functions, including: a confocal scanning light ophthalmoscope (SLO) or a line scan ophthalmoscope (LSO), a cross-sectional tomography (OCT), a fundus camera, even an ultra-high-definition adaptive optics scanning light ophthalmoscope (AOSLO); meanwhile, it also provides a variety of imaging module combinations, such as SLO+OCT, fundus camera+OCT, fundus camera+SLO, or AOSLO+SLO. Therefore, it may be suitable for different and complex application scenarios, and provide real-time fundus imaging and real-time image stabilization.

5) The present invention is based on a fundus retinal surface imaging function, such as a high-precision fundus navigation and target tracking system of SLO or fundus camera, which may ensure that the clinician may easily select a pathological area; meanwhile, it also provides a smart disease diagnosis function (using artificial intelligence technology) to help the clinician to perform the preoperative planning, provide the surgical reference area, and simplify the operation.

6) The present invention employs a data control and data processing system, and thus could analyze preoperative imaging, diagnose the disease condition and record the image data in the database; could combine real-time imaging to facilitate the clinician to confirm that the treatment area is accurate during treatment; and could analyze postoperative imaging to facilitated the clinician to evaluate the surgery, and meanwhile, input the postoperative image in the database for indexing and further application.

7) The laser output adjustment module and laser control module of the present invention may combine fundus image data feedback to perform a smart laser strike, may achieve an accurate strike, use a low-power same-color light for target recognition, and achieve an accurate laser treatment after locking the treatment area, help clinician to operate. The laser treatment device may also automatically adjust a spot size, and an operator may select the spot size according to requirement; a conventional CW laser may be used as a laser source, or a picosecond or femtosecond laser may be used as the light source; when using the femtosecond laser for fundus laser surgery, a photomechanical effect may be used to achieve a purpose of accurate treatment.

### BRIEF DESCRIPTION OF THE DRAWINGS

FIG. 1 is a schematic diagram of a smart treatment system for fundus laser surgery according to an embodiment of the present invention;

FIG. 2 is a principle schematic diagram of a hardware implementation of the imaging stabilization and laser treatment device 1 shown in FIG. 1 of the present invention;

FIG. 3 is a schematic diagram of a typical SLO fast scanning and slow scanning mechanism;

FIG. 4 is a schematic diagram of an implementation of the beam splitting device S1 shown in FIG. 2;

FIG. 5 is a schematic diagram of fundus tracking in a scanning direction of a sawtooth wave implemented by stacking an offset amount on the sawtooth wave;

FIG. 6 is a principle schematic diagram of a mechanical device for controlling the mirror M3 according to an embodiment of the present invention;

FIG. 7 is a schematic diagram of a two-dimensional scanning method for controlling a position of OCT in a scanning space of fundus according to an embodiment of the present invention;

FIG. 8 is a schematic diagram of a design method of a beam splitting device S3 corresponding to an auxiliary module light source according to an embodiment of the present invention;

FIG. 9 is a schematic diagram of a combined mechanical and electronic device for notifying a user and a host control system of whether the current auxiliary module is in imaging mode 2 or laser treatment according to an embodiment of the present invention;

FIG. 10 is a functional block diagram of a smart auxiliary diagnosis system for fundus laser surgery according to an embodiment of the present invention;

FIG. 11 is a schematic diagram of a smart laser treatment according to an embodiment of the present invention, which is used to provide a clinical treatment reference scheme;

FIG. 12 is a schematic diagram of another smart laser treatment according to an embodiment of the present invention, which is used to provide a clinical treatment reference scheme;

FIG. 13 is a schematic diagram of yet another smart laser treatment according to an embodiment of the present invention, which is used to provide a clinical treatment reference scheme.

### DETAILED DESCRIPTION OF THE INVENTION

Hereinafter, the present invention will be further described in detail in connection with the drawings and embodiments of the present invention.

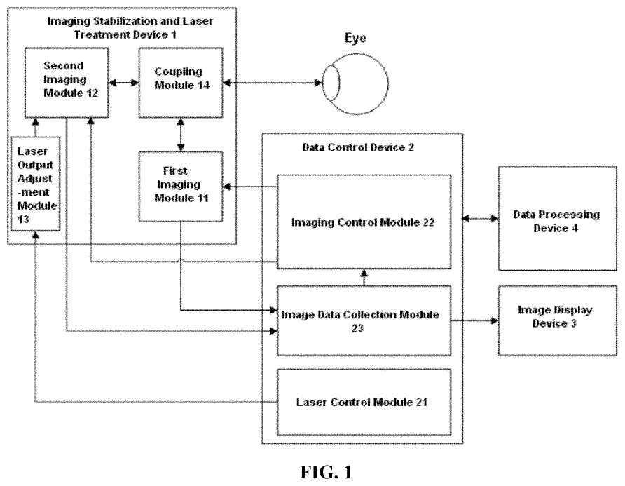

FIG. 1 is a schematic diagram of a smart treatment system for fundus laser surgery according to an embodiment of the present invention.

As shown in FIG. 1, the smart treatment system for fundus laser surgery is also an ophthalmology diagnosis and treatment platform, and mainly includes a imaging stabilization and laser treatment device 1, a data control device 2, and an image display device 3. Preferably, it may further include a data processing device 4.

The imaging stabilization and laser treatment device 1 further includes an imaging diagnosis module 1A and a laser treatment module 1B. As another embodiment, the laser treatment module 1B may be combined with one of imaging modules (i.e., a second imaging module 12); preferably, it may also share hardware with the second imaging module 12 to achieve an objective of cost saving and convenient control.

The laser treatment module 1B includes a laser output adjustment module 13 and a second imaging module 12; the imaging diagnosis module 1A includes a first imaging module 11 and a coupling module 14.

Specifically, in this embodiment, the first imaging module 11 is set as a master module, and correspondingly, the scanning mirrors therein are master scanners. The second imaging module 12 and the laser output adjustment module 13 (used for laser treatment) are configured as slave modules, and correspondingly, the scanning mirrors therein are slave scanners. The first imaging module 11 may be a confocal scanning laser ophthalmoscope (SLO) or a line scanning ophthalmoscope (LSO), or a fundus camera, or an ultra-high-definition adaptive optics scanning light ophthalmoscope (AOSLO). The second imaging module 12 may be an optical coherence tomography (OCT) or SLO. Correspondingly, the first imaging module 11 and the second imaging module 12 support a plurality of imaging module combinations, such as SLO+OCT, fundus camera+OCT, fundus camera+SLO, or AOSLO+SLO.

The laser output adjustment module 13 has a built-in zoom lens for adjusting a laser output dose. It may also control a size of a fundus laser spot by changing a position of the zoom lens to facilitate a clinical operation.

The data control device 2 further includes a laser control module 21, an imaging control module 22 and an image data collection module 23.

Through the data control device 2 and the imaging control module 22, the first imaging module 11 and the second imaging module 12 are controlled in real time. Furthermore, the first imaging module 11, such as the SLO, the LSO, or/and the second imaging module 12, such as the OCT, are used to perform a scanning imaging through a galvanometer.

The data control module 2 realizes a real-time scanning of the fundus by adjusting parameters such as clock signal, amplitude, frequency and the like of the system. Meanwhile, the data control module 2 may also control vibrating optic elements in the first imaging module 11 and the second imaging module 12 simultaneously, and arbitrarily (at an angle) change scanning parameters, such as the size of the image, the frame rate of the image, the brightness and gray scale control of the image, the pixel resolution of the image, and the dynamic range of the image and the like. In addition, the image collection may be performed through a data collection port of the image data collection module 23, and the fundus images of the first imaging module 11 and the second imaging module 12 may be displayed on the image display device 3 in real time to facilitate a clinician to perform an observation and diagnosis in real time.

Preferably, the clinician may analyze the obtained image in real time using the data processing device 4, and provide a relevant reference treatment scheme. For example, the clinician may mark a reference treatment area, provide a reference laser dose standard corresponding to each area, provide a laser spot size corresponding to each area, and so on.

In addition, the imaging stabilization and laser treatment device 1 of an embodiment of the present invention may realize a fundus target tracking and locking function. The specific process is as follows. Through a fundus image information obtained by the first imaging module 11, a human eye motion signal (including motion signal x and y) is calculated in real time and sent to the data control device 2. The data control device 2 outputs a real-time control signal through the imaging control module 22 to change the position of the galvanometer in the second imaging module 12 and lock it with the target in real time, so as to achieve the purpose of real-time target tracking and locking. The real-time control signal may be calibrated in advance to ensure that a change of the galvanometer position is consistent with the actual eye offset.

In an embodiment of the present invention, the laser output adjustment module 13 and the second imaging module 12 of the laser treatment device support sharing a hardware system. The function of fundus imaging and laser treatment may also be realized through a cooperation of a coupler.

The data control device 2 may control the fundus target for imaging and adjust the laser output in the laser output adjustment module 13 in real time through the imaging control module 22 and the laser control module 21 respectively, including adjusting an output power, an output switching, a modulation of output signal, and so on.

The laser control module 21 may use two lasers with similar wavelengths, or the same laser may be used as both the treatment laser and the reference light. In this embodiment, the laser light source may be a 532 nm CW or a femtosecond laser system.

After the laser treatment is finished, the clinician may also observe the fundus image of the patient after the treatment in real time through a display screen of the image display device 3, evaluate the result of the surgery in real time, and support uploading the fundus image of the patient into a database file in the data processing device 4 to facilitate later follow-up observation.

In an embodiment of the present invention, a human eye fundus is taken as an example. The imaging stabilization and laser treatment device 1 composed of the first imaging module 11, the second imaging module 12, and the coupling module 14 may also be used for other different biological tissues, such as gastrointestinal, skin and the like. The following description may still be applied to human fundus as an example.

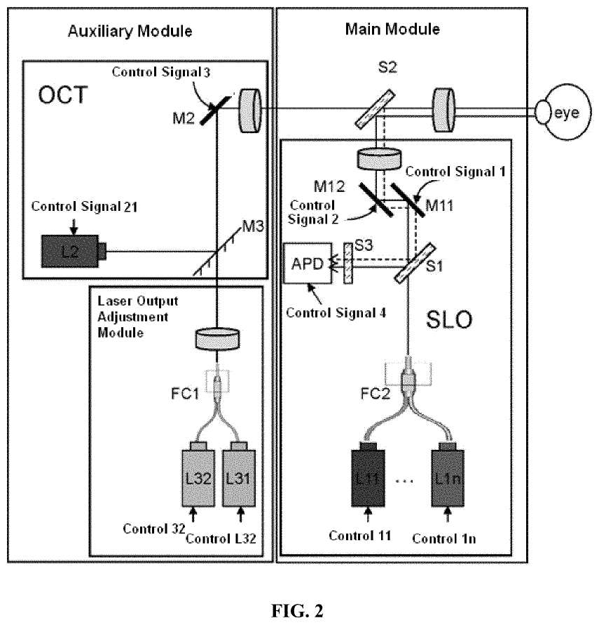

FIG. 2 is a principle schematic diagram of a hardware implementation of the imaging stabilization and laser treatment device 1 shown in FIG. 1 of the present invention.

As shown in FIG. 2, the imaging stabilization and laser treatment device may be used as an independent laser fundus navigation and treatment equipment, or may be combined with another data control device as a complete laser surgery treatment system for clinical application.

In FIG. 2, light sources L11, L12, . . . , L1n are a plurality of imaging light sources controlled (or modulated) by control (signals) 11, 12, . . . , 1n, respectively, for the first imaging module 11 to perform imaging. For example, an infrared light with a wavelength of 780 nm is used for fundus reflection imaging, and a light with a wavelength of 532 nm is used for fundus autofluorescence imaging, or light sources of other wavelengths are used for other forms of fundus imaging. The plurality of imaging light sources may enter an optical system through a fiber coupling device FC2. Any one of the light sources L11 . . . L1n may be controlled (or modulated), such as the control signals of the main module as shown in FIG. 2, that is, control (signal) 11, . . . , control (signal) 1n. The control (or modulation) parameters, including the output power, switching state, and so on, may also be selectively synchronized or unsynchronized with a scanning mirror. The related technology synchronized with the scanning mirror has been described in detail in a previously filed patent application, and will not be repeated herein.

The imaging light sources L11 . . . L1n pass through a beam splitting device S1, pass through a scanning mirror M11 and a scanning mirror M12, and then pass through a beam splitting device S2, and enter the fundus of eye.

A signal returned from the fundus, such as a reflected signal of photoreceptor cell, or a fluorescent signal excited by fundus protein, or other signals returned from the fundus, may be reflected along the same optical path to reach the beam splitting device S1, and then pass through another movable beam splitting device S3 to reach a photodetector, such as an avalanche photodiode (APD). In an embodiment of the present invention, the APD is described to be an example used as the photodetector. The photodetector may also be a photomultiplier tube (PMT), a CMOS, a CCD, or another photodetector device.

In an embodiment of the present invention, each of the above-mentioned photodetectors (such as APD, PMT, CMOS, CCD) is provided with a controllable or programmable gain adjustment mechanism, which may be dynamically adjusted by receiving a program control signal of a system host, so as to adapt different imaging modes. For example, a dynamic adjustment may be made through the control signal 4 shown in FIG. 2.

The set of scanning mirrors M11 and M12 shown in FIG. 2 are mainly used for orthogonal scanning of the fundus imaging position, and the scanning axes of the scanning mirrors M11 and M12 are usually 90 degrees.

In the case of the first imaging module 11 corresponding to the SLO, the scanning mirror M11 may be a fast resonant scanner. A typical practical application scenario is to configure the scanning mirror M11 to scan in a horizontal direction and configure M12 that is a slow linear scanning mirror to scan in a vertical direction. In general, the orthogonal scanning directions of the scanning mirrors M11 and M12 support scanning in any direction of 360 degrees in a two-dimensional space. In an embodiment of the present invention, the scanning mirror M11 employs a CRS8k fast resonant scanner of Cambridge Technology. In another application system, a CRS12k or another type of fast resonant scanner may also be employed.

In the case of the first imaging module 11 corresponding to the SLO, the scanning mirror M12 in an embodiment of the present invention may be implemented by one two-dimensional steering mirror or two one-dimensional steering mirrors. In the actual optical-mechanical system of the present invention, the scanning mirror M12 employs a set of two-dimensional scanning mirrors 6220H (or 6210H) of Cambridge Technology. A first axis of 6220H, i.e., a slow scanning axis, is orthogonal to a scanning direction of a fast scanning axis of the M11; a second axis of 6220H does not participate in scanning but is only used for target tracking, and is parallel to a scanning axis of M11.

In the above case of corresponding to the SLO, a scanning field of the scanning mirror M11 as a fast resonant scanner is controlled by the system host or manually.

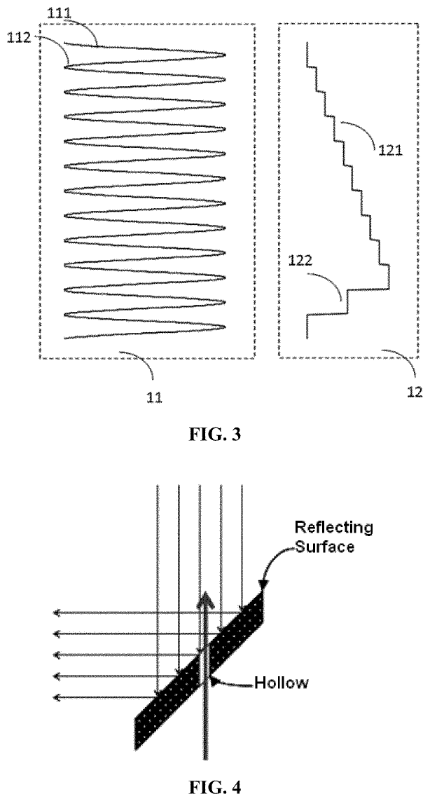

In the above embodiment, the scanning motion track of the M12 orthogonal to the M11 is a triangular wave. The scanning parameters such as the amplitude and frequency of the triangle wave, the rising period and the falling period of the triangle wave, and so on are controlled by the system host. The amplitude of the triangle wave determines the size of the field of view in the slow scanning direction, and the frequency of the triangle wave determines the frame rate of the image system (referring to FIG. 3).

FIG. 3 is a schematic diagram of a typical SLO fast scanning and slow scanning mechanism. Each time the fast resonant scanner scans a cycle, the slow mirror linearly increases by one step.

As shown in FIG. 3, normally, each time the fast (resonant) scanning of the SLO completes a sine (or cosine) period 11, the slow (linear) scanning moves by one step 12 in the orthogonal direction. In this way, the image frame rate (fps), the resonance frequency (0 of the fast scanning mirror, and the number of lines (N) contained in each frame of the image (usually representing the maximum image height, in special cases may also be used as the image width) satisfy the following relationship:

f=fps·N

In the above equation, N includes all the scanning lines 121 and 122 in the part of FIG. 3, in which 121 is the rising period of the sawtooth wave, and 122 is the falling period.

The SLO image generally does not include the part 122 of FIG. 3, because the image during the 122 period and the image during the 121 period have different pixel compression ratios. The SLO image is generally only obtained from the part 121 of FIG. 3.

The function of the beam splitting device S1 shown in FIG. 2 is to transmit all incident light from the coupling device FC2, but reflect all signals from the fundus to the APD. One implementation mode is to dig a hollow cylinder at the axis of the S1 to allow the incident focused light from FC2 to pass through, but reflect all the expanded light from the fundus to the photodetector APD, as shown in FIG. 4, which is a schematic diagram of an implementation of the beam splitting device S1 shown in FIG. 2.

As mentioned above, the scanning mirror M12 of FIG. 2 has two independent motion axes. The first motion axis is orthogonal to the motion (scanning) axis of the M11, and the second motion axis is parallel to the motion (scanning) axis of the M11.

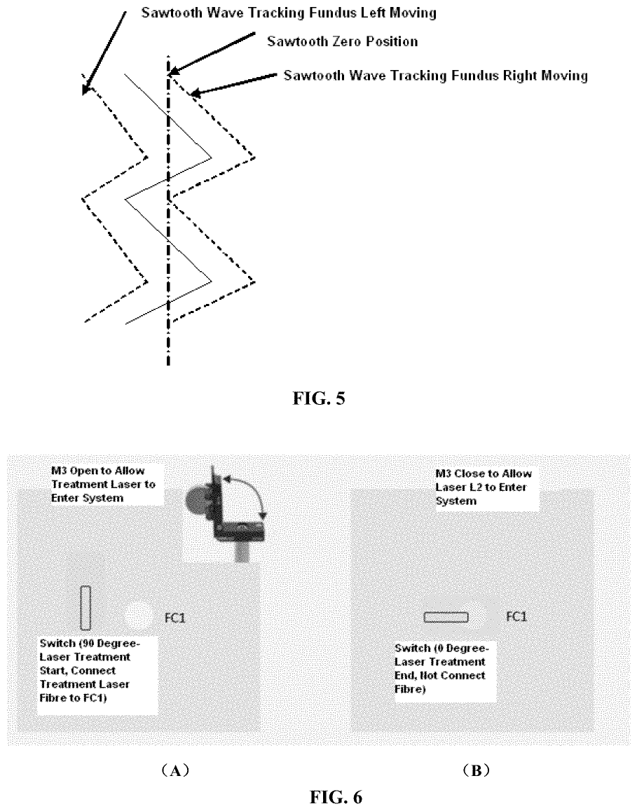

The motion (scanning) axis of the scanning mirror M12 is orthogonal to the motion axis of the M11, which may receive two signals from the system host: one is the sawtooth wave shown in FIG. 3 (such as 121 and 122), and the other is a translation signal superimposed on the sawtooth. The sawtooth wave is used to scan the fundus to obtain the fundus image, and the translation signal is used to optically track the eyeball motion of the fundus in the scanning direction of the sawtooth wave, as shown in FIG. 5.

FIG. 5 is a schematic diagram of fundus tracking in a scanning direction of a sawtooth wave implemented by stacking an offset amount on the sawtooth wave.

As shown in FIG. 5, when a target (such as the eyeball) is at a certain reference time point, that is, a reference surface of a tracking algorithm, the scanning center of the sawtooth wave is at a relative zero position. When the eyeball starts to move relative to the reference surface, a control host adjusts an offset amount of the sawtooth wave in real time to track the position of the fundus relative to this reference surface.

The system control host mentioned above may be a PC provided with a corresponding control program module, or a device including a field programmable gate array (FPGA), or a device including a digital signal processor (DSP), or a device that uses another type of electronic signal processor, or a combined device including these hardware.

For example, in an embodiment of the present invention, the control device uses an Intel PC (Intel i7) machine equipped with a nVidia graphic processing unit (GPU), such as GTX1050, which is used to calculate an eyeball motion signals (x, y, 0), and then through a Xilinx FPGA (considering the cost factor, an embodiment of the present invention uses a device ML507 of Virtex-5 or SP605 of Spartan 6; in the future, may use other more powerful but also more expensive latest series of FPGA devices such as Virtex-6, Virtex-7, Kintex-7, Artix-7 and so on, or FPGA devices from other manufacturers such as Altera), by digitally synthesizing the y part of (x, y, 0) into the signal form of FIG. 5, and then sending it to a digital-to-analog converter (DAC), such as a DAC5672 of Texas Instruments, controls the first motion axis of the scanning mirror M12.

The signal in FIG. 5 may also be realized by an analog synthesis. In this case, the sawtooth wave in FIG. 5 is generated by a first DAC to generate a first analog signal. The offset amount in FIG. 5 is also the y component of (x, y, θ), and a second analog signal is generated by a second DAC. The two analog signals are synthesized by an analog signal mixer, which is finally sent to the first motion axis of the scanning reflector M12.

The x of the signal (x, y, θ) is an analog signal generated by another separate DAC and sent to the second motion axis of the M12 to track the motion of the eyeball on the second motion axis. In an embodiment of the present invention, the second motion axis of the scanning mirror M12 is parallel to the scanning axis of the M11.

The translation part (x, y) of the above-mentioned eyeball motion signal (x, y, θ) has two orthogonal motion axes of the M12 to realize a closed-loop optical tracking. The rotating part (θ) of the first imaging module 11 is implemented by a digital tracking in an embodiment of the invention, but it may also be implemented by an optical or/and mechanical closed-loop tracking in the future. The related technology of the optical or/and mechanical tracking of the rotating part (θ) is described in detail in the U.S. Pat. No. 9,775,515.

There are two key terms that are frequently switched mentioned in the embodiments of the present invention: fundus tracking and eyeball tracking. In the technology related to the present invention, fundus tracking and eyeball tracking are one concept. In clinical application, most of the physical motions come from the eyeball, and the motion of the eyeball causes the fundus image obtained by the imaging system to change randomly in space over time. The equivalent consequence is that at any time point of the imaging system, different images are obtained from different fundus positions, and the observed result is that the images jitter randomly over time. The tracking technology in an embodiment of the present invention is to capture the eyeball motion signal (x, y, θ) in real time through the fundus image in the imaging system, and then feedback (x, y) to the M12 in FIG. 2. Thus the scanning spaces of two scanning mirrors (M11 and M12 orthogonal to the direction of M11) are locked in a pre-defined fundus physical space, thereby realizing accurate fundus tracking and stabilizing the random change of the fundus image in space over time.

The imaging mode in FIG. 2 (corresponding to a main module) constitutes a complete closed-loop control system for high-speed real-time tracking of fundus position. This part of the technology is described in detail in two U.S. Pat. Nos. 9,406,133 and 9,226,656.

The imaging mode 2 of FIG. 2, that is, the “slave L2-M3-M2-S2-fundus” on the left, corresponds to the imaging mode 1 (the main module) shown in FIG. 1. A typical application is an application of optical coherence tomography (OCT) imaging technology.

In FIG. 2, “L31/L32-M2-S2-Fundus” corresponds to the fundus laser treatment device described in FIG. 1. The functional realization of the OCT and the fundus laser treatment is described in detail below.

M3 is a movable mirror. The movement manner may be mechanical, electronic, or a combination thereof. The movable part of the mirror M3 may also be replaced by a beam splitting device.

In an embodiment of the present invention, the state of the mirror M3 is controlled mechanically. The state of the M3 entering into/exiting from the optical system is determined by the state of the coupling device FC1 in FIG. 2. When the light source L31/L32 is connected to the optical system through the FC1, the M3 is pushed out of the optical system, and the light of the L31/L32 directly reaches the mirror M2. When the FC1 is not connected to the optical system, the M3 is disposed in the position shown in FIG. 2 to reflect the light from the L2 to the mirror M2. The principle that the movable mirror M3 is mechanically controlled by the FC1 is shown in FIG. 6.

FIG. 6 is a principle schematic diagram of a mechanical device for controlling the mirror M3 according to an embodiment of the present invention.

As shown in FIG. 6, in this mechanical device, the M3 is pushed out or put into the optical system according to an insertion and withdrawal mechanism of the FC1. A switch is connected to the foldable frame through a connecting rod. When the switch is at 90 degree as shown in the figure, the frame is opened and the FC1 interface is also opened, allowing entry of a treatment laser, as shown in FIG. 6A. When the switch is closed, as shown in FIG. 6B, at 0 degree, the FC1 interface is closed. At this time, the treatment laser cannot enter. Meanwhile, the foldable frame returns to the original position (refer to FIG. 2), and the imaging laser L2 may be reflected to enter the system.

The function of the mirror M3 is to allow the user to select one of the functions of the imaging mode 2 or the fundus laser treatment in the slave module.

When realizing the OCT imaging, that is, the imaging mode 2 shown above, the M3 is disposed in the optical path of “L2-M3-M2-S2-fundus” shown in FIG. 2, so that the light source L2 reaches the fundus.

In the case of the imaging mode 2 shown in FIG. 2, the light of the L2 reaches the M2 through the M3. The M2 is a two-dimensional scanning mirror, which may be controlled by a fast steering mirror with two independent orthogonal control axes and a single reflective surface (such as S334.2SL of Physik Instrumente), or by two one-dimensional steering mirrors for orthogonal scanning. The latter case is used in the present invention, and a combination of two 6210H mirrors of Cambridge Technology of the United States is used.

In an embodiment of the present invention, the M2 in FIG. 2 has a plurality of functions. In the case of the imaging mode 2 shown in FIG. 2, the system host generates an OCT scan signal to control the scanning mode of the M2, thereby controlling the two-dimensional imaging space of the L2 in the fundus.

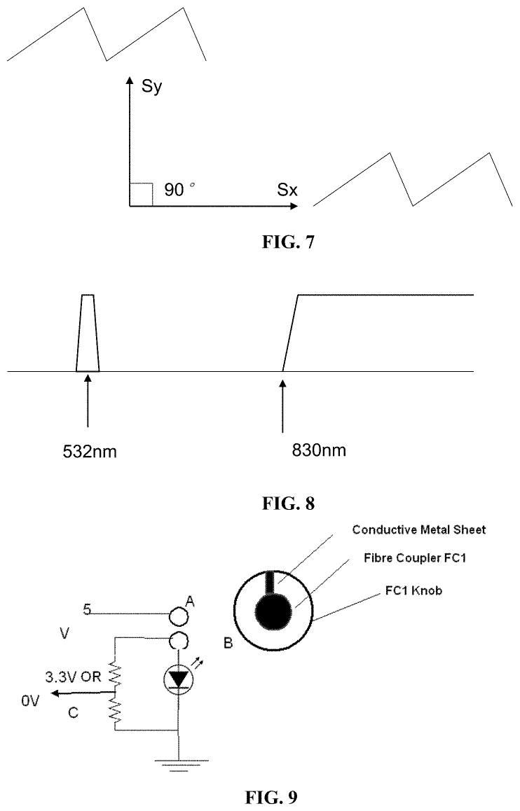

In an embodiment of the present invention, the system host program generates a set of orthogonal scan control bases Sₓ and S_{y} as shown in FIG. 7 by controlling an FPGA. Here Sₓ and S_{y} are vectors with a direction.

FIG. 7 is a schematic diagram of a two-dimensional scanning method for controlling a position of OCT in a scanning space of fundus according to an embodiment of the present invention.

The system host program controls the two scan bases of the FPGA (as shown in FIG. 7) to multiply their respective amplitudes (Aₓ and A_{y}) and positive/negative signs to achieve the OCT in any direction of 360 degree in the fundus. By specifying the two-dimensional field of view size scanning, it may be expressed by the following relationship:

OCT scan=SₓAₓ+S_{y}A_{y};

The parameters Aₓ and A_{y} are also vectors with a sign (or) direction; SₓAₓ+S_{y}A_{y} may realize the OCT in any direction of the 360-degree two-dimensional fundus space, and perform a scanning of any field of view allowed by the optical system.

The light from the light source L2 passes through the mirror M3, the scanning mirror M2, and then reaches the fundus through the beam splitting device S3. In an embodiment of the present invention, the L2 is an imaging light source with a wavelength of 880 nm, the light source L31 has a wavelength of 561 nm, and the light source L32 has a wavelength of 532 nm. Correspondingly, the design of the light splitting device S3 needs to be changed differently for different auxiliary module light sources. One way is to customize a different light splitting device S3 for a different slave module light source and dispose it at the S3 position in FIG. 2, as shown in FIG. 8.

FIG. 8 is a schematic diagram of a design method of a beam splitting device S3 corresponding to an auxiliary module light source according to an embodiment of the present invention.

As shown in FIG. 8, the beam splitting device S3 transmits 90%-95% and reflects 5%-10% of a light at 532 nm and above 830 nm, and transmits 5%-10% and reflects 90%-95% of a light in other wavelength bands.

Referring to FIG. 2, the light source L31 in the auxiliary module is an aiming light for laser treatment. The aiming light reaches the fundus, and a light spot reflected from the fundus is received by the APD of the first imaging module 11, and a light spot generated by L31 is superimposed on the SLO image. This spot position indicates that the treatment light L32 will have a nearly uniform spatial position on the fundus. The degree of overlap of the light sources L31 and L32 on the fundus depends on the transverse chromatic aberration (TCA) produced by the two wavelengths of 532 nm and 561 nm on the fundus.

In an embodiment of the present invention, as for the light with wavelengths of 532 nm and 561 nm, the TCA generated on the fundus will not exceed 10 microns. In other words, after the 561 nm aiming light of the L31 is aimed at the striking position of the fundus, the wrong position of the 532 nm treatment light of the L32 will not exceed 10 microns.

The power of the aiming light of the L31 reaching the fundus is generally below 100 microwatts, and the power of the treatment light of the L32 reaching the fundus may be several hundred milliwatts or more. The signal amplitude reflected by the L31 from the fundus to the APD is close to the image signal amplitude of the SLO, but the 532 nm high-power therapeutic light still has a considerable signal reflected to the SLO through the beam splitting device S3.

In order to prevent the treatment light from turning on the fundus laser strike, the 532 nm signal returned from the fundus reaches the SLO and impacts the APD and causes the APD to be overexposed. In the device of an embodiment of the present invention, the beam splitting device S3 is disposed in front of the APD. The S3 reflects all light below 550 nm and transmits all light above 550 nm to protect the APD.

The beam splitter S3 in FIG. 3 is movable, and the moving state is opposite to that of the M3. When the coupler FC1 is connected to the optical system, the S3 is also connected to the optical system; when FC1 is not connected to the system, the S3 is pushed out of the optical system. Connecting and pushing out the S3 to and of the optical system may be mechanical, electronic, or a combination of thereof. In an embodiment of the present invention, a mechanical method is employed, as shown in FIG. 6.

As described above, the auxiliary module integrates two functions, namely, a laser imaging and an image stabilization, and the laser treatment is implemented using the second imaging module 12 and the laser output adjustment module 13.

Switching between the above two functions is achieved by changing the position of the M3. When the M3 is disposed in the optical system, the second imaging module 12 is activated and the laser treatment device does not operate. When the M3 is pushed out of the optical system, the laser treatment function is activated, and the second imaging module 12 does not operate at this time.

The above is a description of an engineering implementation involving the second imaging module 12. An engineering realization of the laser treatment function involved in an embodiment of the present invention will be described below.

Referring to FIG. 6, the positions of the M3 and the S3 in the optical system is controlled by a position of a knob mounted on the coupling device FC1 to realize the function of dynamically switching the imaging mode 2 and the laser clinical treatment. Another function of the FC1 knob is to connect and disconnect one or more electronic devices to prompt the user and the system host control program which of the two functions should be performed.

FIG. 9 is a schematic diagram of a combined mechanical and electronic device for notifying a user and a host control system of whether the current auxiliary module is in imaging mode 2 or laser treatment according to an embodiment of the present invention.

As shown in FIG. 9, the device controls an LED indicating lamp and provides a high/low level signal to the electronic hardware through a conductive metal sheet mounted on the FC1 knob to inform the user and the host control system of whether the current slave module operates in an imaging and image stabilization mode or a laser treatment mode.

In a default configuration, A and B are disconnected, the LED is off, and point C outputs a 0V voltage or a low level. In an embodiment of the present invention, point C is connected to the FPGA to detect whether an input terminal is a low level (0V) or a high level (3.3V or 2.5V), so as to control the software to automatically switch to the imaging and image stabilization mode or the laser therapy mode.

When the FC1 knob is rotated by 90 degree (or another angle, but consistent with FIG. 6), the conductive metal sheet connects A and B to make the LED light, and at the same time point C is pulled up to a high level. This control program automatically switches the laser treatment function.

When configured for the imaging and image stabilization mode, the entire system may also be used as only the imaging mode 1, such as only the SLO/SLO imaging, without OCT. This operation manner may be achieved through the system host control program.

In the operation mode of the imaging mode 1 combined with the laser treatment, the control M2 in FIG. 2 combines a variety of laser strike modes, including: 1) a single-point strike mode; 2) a regular space area array strike mode; 3) a customized multi-point strike mode in irregular space area.

The single-point strike mode is that the user uses a real-time image of the imaging mode 1 to determine the laser strike position in the pathological area. After aiming at the target with the aiming light, the user starts the treatment light to strike the target with parameters such as a laser dose, an exposure time, and so on set in advance.

The regular space area array strike mode is a combination of the single-point strike mode and the scanning mode of the imaging mode 2, allowing the user to define the parameters such as the laser dose for each position, then start the treatment light, and strike the predetermined targets one by one at equally spaced time intervals.

The customized multi-point strike mode in irregular space area is a completely free strike mode. The user customizes the parameters such as the laser dose, the exposure time and so on of any strike position in the pathological zone, and then strikes the predetermined targets one by one.

Preferably, in order to precisely control the dose of the laser reaching the strike target, in an embodiment of the present invention, a beam splitting device is used to send a part of the light obtained from the treatment light L32 to a power meter. The control program reads the value of the power meter in real time, and dynamically adjusts the laser dose of the L32 power reaching the target to a preset value.

Preferably, in order to precisely control the exposure time of the laser striking the target, in an embodiment of the present invention, an FPGA hardware clock is used to control the on and off states of the L32. A control method may be implemented through a real-time operating system, such as Linux. Another control method may be implemented by installing real-time control software (Wind River) on a non-real-time operating system such as Microsoft Windows; yet another control method may be to control by a timer on a completely non-real-time operating system such as Microsoft Windows.

All the functions of the above auxiliary modules, including the imaging and image stabilization and the laser treatment functions, may be supported by the real-time target (fundus) tracking and real-time image stabilization technology of the main module.

After the closed-loop fundus tracking function of the main module is activated, the host control software displays a stable SLO/LSO image in real time. In an embodiment of the present invention, the spatial resolution of the image stabilization technology is approximately ½ of the lateral optical resolution of the imaging module 1. The stabilized real-time SLO/LSO image allows the user to conveniently locate the fundus space position to be processed by the auxiliary module.

The fundus tracking of the main module is a closed-loop control system. After the fundus tracking function is activated, an instruction of the master module for controlling the tracking mirror M12 is sent to the M2 of the slave module according to the pre-calibrated mapping relationship. Therefore, the light coming from the L2 or the L31/L32 may be locked to a predetermined fundus position with considerable accuracy after reaching the fundus through the M2. A core technology herein is to use the closed-loop control instruction of the main module to drive an open-loop tracking of the auxiliary module.

The spatial mapping relationship between the M12 and M2, that is, how to convert the control instruction (x, y, θ) of the M12 into the control instruction (x′, y′, θ′) of the M2 depends on the design of the optical system.

Here, the (x, y, θ) and the (x′, y′, θ′) have the following relationship:

(x′,y′,θ′)=f(x′,y′,θ′;x,y,θ)(x,y,θ)

wherein, (x′, y′, θ′; x, y, θ) may be realized by a calibration of the optical system.

The core technology is in that the closed-loop control instruction of the master module drives the open-loop tracking of the slave module, which is an M12 closed-loop and M2 open-loop optical tracking.

Referring to FIG. 7, as shown in the equation “OCT scan=SₓAₓ+S_{y}A_{y}”, the scanning mirror M2 of the auxiliary module may perform an optical scanning in any direction of 360 degree in the two-dimensional space. Therefore, the auxiliary module M2 is an open-loop optical tracking of the three variables (x′, y′, θ′) in the above equation, although the main module only has a closed-loop optical tracking of translation (x, y) and a digital tracking of rotation θ.

The closed-loop tracking accuracy of the main module and the calibration accuracy of the above equation determine the open-loop tracking accuracy of the light from the auxiliary module to the fundus, or the accuracy of target locking. In the most advanced technologies available, the closed-loop optical tracking accuracy of the main module is equivalent to the optical resolution of the imaging system of the main module, about 15 microns, and the open-loop optical tracking accuracy of the auxiliary module may reach ⅔-½ of the closed-loop optical tracking accuracy of the main module, or 20-30 microns. It is necessary to emphasize that in different system devices, these accuracies will vary differently.

The present invention is mainly applied to ophthalmology, and the targeted cases are diabetic retinal degeneration, age-related macular degeneration and the like. The fundus laser treatment technology provided by the present invention supports a smart automatic fundus diagnosis and treatment solution, and also provides a material basis for an one-stop diagnosis and treatment service in the future.

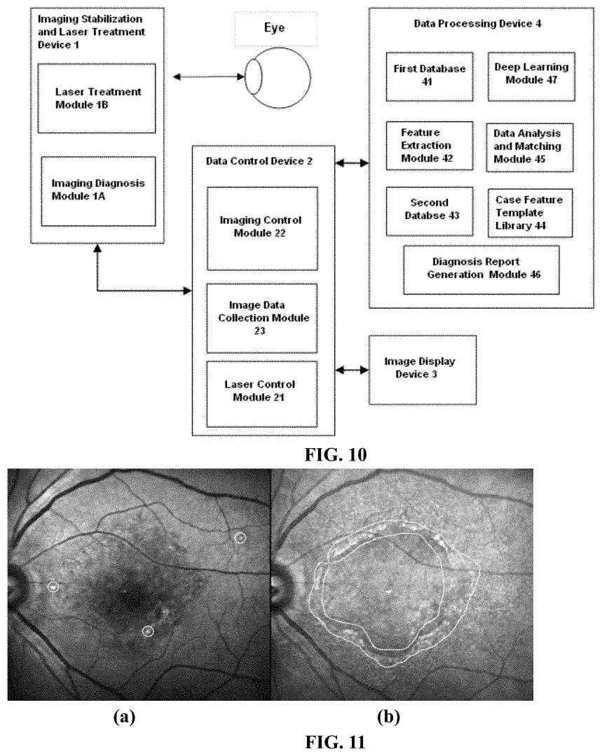

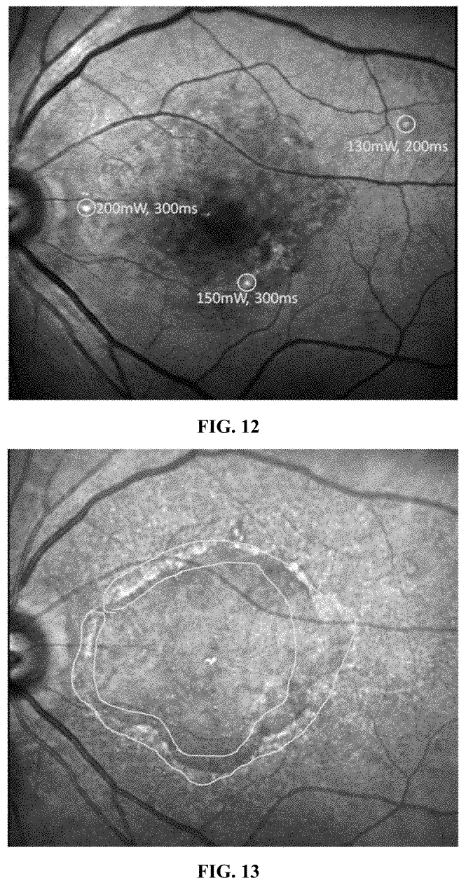

FIG. 10 is a functional block diagram of a smart auxiliary diagnosis system for fundus laser surgery according to an embodiment of the present invention. FIG. 11 is a schematic diagram of a smart laser treatment according to an embodiment of the present invention, which is used to provide a clinical treatment reference scheme; FIG. 12 is a schematic diagram of another smart laser treatment according to an embodiment of the present invention, which is used to provide a clinical treatment reference scheme; FIG. 13 is a schematic diagram of yet another smart laser treatment according to an embodiment of the present invention, which is used to provide a clinical treatment reference scheme.

As shown in FIG. 10, the smart auxiliary diagnosis system for fundus laser surgery mainly uses the imaging stabilization and laser treatment device 1 to collect high-definition fundus image data (including image and video, stored in the first database 41) acquired at any angle and with various imaging methods, processes and analyzes the fundus image by the data processing device 4, for example, the feature extraction module 42 extracts disease feature data in the fundus image, and the data analysis and matching module 45 perform a comparison operation to compare with the disease feature data stored in the known case feature template library 44, and the result of the matching operation is stored in the second database 43. If the matching degree exceeds the set threshold, the corresponding auxiliary diagnosis conclusion is provided, and then the auxiliary diagnosis report is generated by the diagnosis report generation module 46. The main content of the auxiliary diagnosis report includes the preoperative diagnosis scheme, the intraoperative target determination scheme, and the postoperative treatment effect prediction result.

Preferably, it further includes the deep learning module 47, which is used to perform a large amount of data training based on the collected fundus image data of the patient in combination with the disease feature data extracted from the fundus image, and automatically perform the data analysis and matching operation (using data fuzzy matching algorithm), and provide the matching operation result that may be referenced by the medical expert. Finally, 1) it matches the result of the matching operation with a matching degree greater than the set threshold with the case in the case feature template library and register it as a case; 2) it matches the result of the matching operation with a matching degree less than the set threshold (may be a new discovered case or not) confirmed by the medical expert, writes the case feature data corresponding to the fundus image into the new case feature template and inputs it into the case feature template library 44, that is, updates the case feature template library.

As another embodiment, the deep learning module 47 may also be disposed in a cloud server, and the fundus image data of the patient transmitted from another smart auxiliary diagnosis system for fundus laser surgery through the Internet may be used as training data. In combination with the latest disease feature data extracted from the existing known fundus images, the deep learning module 47 may perform a large amount of data training, and automatically perform the data analysis and matching operation (using parallel, multi-dimensional data fuzzy matching algorithms) to provide the matching operation result for the medical expert's reference.

As shown in FIG. 11, an example of multi-wavelength synchronous imaging according to an embodiment of the present invention is shown to more accurately locate the case area and then realize a laser strike. Clinically, a single wavelength cannot accurately locate all pathological areas. The smart auxiliary diagnosis system for fundus laser surgery of an embodiment of the present invention employs different wavelengths for synchronous imaging, as different cells and different proteins have different sensitivities to lights of different wavelengths. As shown in FIG. 11a, the three pathological areas indicated by the circles in the figure are not obvious in FIG. 11b, and the pathological areas in the white area in FIG. 11b are not obvious in FIG. 11a. Therefore, a significant function of the multi-wavelength synchronous imaging is to allow the clinician to dynamically observe the pathological areas during the imaging process to achieve a real-time manual or semi-automatic laser strike on the pathological areas.

One function of the multi-wavelength synchronous imaging is to allow the clinician to extract a typical multi-wavelength image from the image database of the software after completing the fundus imaging, as shown in the left and right figures in FIG. 11a and FIG. 11b. Thereafter, the clinician may more accurately identify and edit the pathological areas offline, and reasonably arrange the laser strike treatment scheme. One method is shown in FIG. 12. The clinician configures the laser strike dose, the exposure time, and other parameters for each area according to the conditions of the pathological areas. After configurating, as shown in FIG. 12, the image for the pathological areas is imported into the software system, and the image is used as the reference image for tracking to realize a fully automatic or semi-automatic laser strike treatment.

Another function of multi-wavelength synchronous imaging is to allow the clinician to extract a typical multi-wavelength images from the image database of the software after completing the imaging, as shown in the left and right figures in FIG. 11a and FIG. 11b. Another method is shown in FIG. 13. The clinician configures an array laser strike on an entire area according to the conditions of the pathological areas. The software allows the user to configure the laser dose, the exposure time, and other parameters. After configuring, the image for the pathological areas as shown in FIG. 13 is imported into the software system, and the image is used as the reference image for tracking to realize an automatic or semi-automatic array laser strike.

It should be noted that the above embodiments only take two wavelengths as an example for description, and there may be synchronous imaging of more wavelengths in an actual situation. There are mature technologies in existing industrial lasers for controlling the laser exposure dose and the exposure time. For example, an acousto-optic modulator may simultaneously control the laser output power or the exposure dose (analog control), and the switching state of the laser (digital control). The control signals of the present invention come from an FPGA, which may control the switching state of the laser up to nanosecond precision on electronic hardware, and the precision of laser power output is up to the tolerance of the manufacturer (usually in the range of tens of milliseconds to hundreds of nanoseconds).

The above are only the preferred embodiments of the present invention, and are not used to limit the protection scope of the present invention.

1. A smart auxiliary diagnosis system for fundus laser surgery, comprising an imaging stabilization and laser treatment device (1), a data control device (2), and an image display device (3); characterized by further comprising a data processing device (4), wherein:
the data processing device comprises a first database (41), a feature extraction module (42), a data analysis matching module (43), a case feature template library (44), a second database (43), and a diagnosis report generation module (46); the first database (41) is used to store high-definition fundus image data collected by the imaging stabilization and laser treatment device (1) at any angle and with various imaging methods; disease feature data in the fundus image is extracted by the feature extraction module (42), and compared with known disease feature data stored in the case feature template library (44) by a comparison operation using the data analysis matching module (45), and a matching operation result is stored in the second database (43), if a matching degree exceeds a set threshold, a corresponding auxiliary diagnosis conclusion is provided, and then an auxiliary diagnosis report is generated through the diagnosis report generation module (46).

2. The smart auxiliary diagnosis system for fundus laser surgery according to claim 1, characterized in that the imaging stabilization and laser treatment device (1) comprises:
an imaging diagnosis module for obtaining a reflection signal returned from the fundus at any angle in real time or/and obtaining image data of the fundus;
a laser treatment module for tracking and locking a fundus target in real time, and automatically adjusting a laser dose output.

3. The smart auxiliary diagnosis system for fundus laser surgery according to claim 2, characterized in that the imaging diagnosis module supports one or more of a confocal scanning laser ophthalmoscope SLO, a line scanning ophthalmoscope LSO, a fundus camera, or an adaptive optics scanning light ophthalmoscope AOSLO.

4. The smart auxiliary diagnosis system for fundus laser surgery according to claim 2, characterized in that the imaging diagnosis module further supports a combination of a plurality of imaging forms, including one or more of SLO+OCT, fundus camera+OCT, fundus camera+SLO or AOSLO+SLO.

5. The smart auxiliary diagnosis system for fundus laser surgery according to claim 1, characterized in that the smart auxiliary diagnosis system for fundus laser surgery further comprises a deep learning module (47) for performing a large amount of data training by combining the collected fundus image of a patient with the disease feature data extracted from the fundus image, and obtaining the matching operation result for a medical expert's reference by automatically performing a data analysis matching operation.

6. The smart auxiliary diagnosis system for fundus laser surgery according to claim 5, characterized by further comprising: processing the matching operation result for the medical expert's reference:
matching the matching operation result of which the matching degree is greater than the set threshold with a case in the case feature template library and register it as a case; or,
writing case feature data corresponding to the fundus image with the matching operation result of which the matching degree is less than the set threshold confirmed by the medical expert into a new case feature template for inputting into the case feature template library (44), that is, updating the case feature template library.

7. The smart auxiliary diagnosis system for fundus laser surgery according to claim 1, characterized in that contents of the auxiliary diagnosis report include a preoperative diagnosis scheme, an intraoperative target determination scheme, and a postoperative treatment effect prediction result.

8. A smart auxiliary diagnosis method for fundus laser surgery, characterized by comprising the following steps:
A. collecting high-definition fundus image data at any angle and with various imaging methods a using imaging stabilization and laser treatment device (1), and storing it in a first database (41) of a data processing device (4);
B. extracting disease feature data in the fundus image by a feature extraction module (42), and perform a comparison operation using a data analysis matching module (45) to obtain a comparison result;
C. matching the comparison result with the disease feature data stored in known case feature template library (44), and storing a matching operation result in a second database (43);
D. if a matching degree exceeds a set threshold, providing a corresponding auxiliary diagnosis conclusion and then generating an auxiliary diagnosis report by a diagnosis report generation module (46).

9. The smart auxiliary diagnosis method for fundus laser surgery according to claim 8, characterized by, after step D, further comprising:
E. by a deep learning module (47), performing a large amount of data training by combining the collected fundus image of a patient with the disease feature data extracted from the fundus image, and obtaining the matching operation result for a medical expert's reference by automatically performing a data analysis matching operation.

10. The smart auxiliary diagnosis method for fundus laser surgery according to claim 9, characterized in that step E further comprises:
E1. matching the matching operation result of which the matching degree is greater than the set threshold with a case in the case feature template library and register it as a case; or,
E2. writing case feature data corresponding to the fundus image with the confirmed matching operation result of which the matching degree is less than the set threshold into a new case feature template for inputting into the case feature template library (44), that is, updating the case feature template library.

11. The smart auxiliary diagnosis system for fundus laser surgery according to claim 2, characterized in that the laser treatment module comprises a laser output adjustment module and a second imaging module, the imaging diagnosis module comprises a first imaging module and a coupling module, through a fundus image information obtained by the first imaging module, a human eye motion signal is calculated in real time and is sent to the data control device;
the data control device comprises a laser control module, an imaging control module and an image data collection module, the imaging control module outputting a real-time control signal to control vibrating optic elements in the first imaging module and to change a position of a galvanometer in the second imaging module, so as to lock the second imaging module with the target in real time; a closed-loop control instruction of the first imaging module is provided to drive an open-loop control of the second imaging module; the laser control module is disposed to adjust laser output in the laser output adjustment module; the image data collection module is disposed to collect fundus images of the first imaging module and the second imaging module and to send the fundus images to image display device, such that the fundus images are displayed in real time.
